(19) United States
(12) Patent Application Publication
(10) Pub. No.: US 2024/0035091 A1
(43) Pub. Date: Feb. 1, 2024

(54) GENE SIGNATURE AND PREDICTION OF LUNG CANCER RESPONSE TO ADJUVANT CHEMOTHERAPY

(71) Applicant: THE BOARD OF REGENTS OF THE UNIVERSITY OF TEXAS SYSTEM, Austin, TX (US)

(72) Inventors: Yang XIE, Dallas, TX (US); Guanghua XIAO, Dallas, TX (US)

(73) Assignee: THE BOARD OF REGENTS OF THE UNIVERSITY OF TEXAS SYSTEM, Austin, TX (US)

(21) Appl. No.: 18/265,571

(22) PCT Filed: Dec. 8, 2021
(86) PCT No.: PCT/US2021/062436

§ 371 Date: Jun. 16, 2022

Related U.S. Application Data
(60) Provisional application No. 63/122,606, filed on Dec. 8, 2020.

(51) Int. Cl. C12Q 1/6886; G16B 25/10; G16H 20/10; G16H 50/30

(52) U.S. Cl. CPC C12Q 1/6886; G16B 25/10; G16H 20/10; G16H 50/30; C12Q 2600/118; C12Q 2600/158; C12Q 2600/106

(57) ABSTRACT

Provided herein is a 7-genes signature, an algorithm to evaluate a patient's risk score, and methods of use thereof. The methods provided herein include methods of use of a risk score to predict lung cancer patient survival and lung cancer patient likelihood to respond to adjuvant chemotherapy.

### PRIORITY CLAIM

This application claims priority pursuant to 35 USC § 119(e) to U.S. provisional patent application No. 63/122,606 filed Dec. 8, 2020. The aforementioned application is hereby incorporated by reference as though fully set forth.

### BACKGROUND

Lung cancer is the leading cause of death from cancer for both men and women in the United States and in most other parts of the world; and has a 5-year survival rate of approximately 15%. Chemotherapy is the cornerstone of treatment of lung cancer patients; however, the response to standard chemotherapy in lung cancer varies widely among patients, therefore the routine use of chemotherapy is not justified for all patients with resected lung cancer. Accordingly, it is of substantial clinical importance to be able to predict who will benefit from chemotherapy before starting treatment.

Although a large number of cancer biomarkers have been reported, few have been translated into real clinical tools. The major bottleneck in translating biomarker discovery to improve patient outcomes is the availability of accurate prediction algorithms and clinical assays that will allow treatments to be tailored to an individual's needs. An accurate predictive algorithm for response to chemotherapy, together with a reliable clinical assay to measure gene expression from both fresh frozen and formalin-fixed paraffin-embedded tumor samples, will have an immediate impact on patient care in lung cancer; however, there is still an unmet need for such tool.

### SUMMARY

An embodiment provides a method of predicting response to chemotherapy of a human subject diagnosed with lung cancer comprising determining a seven gene signature that predicts response to chemotherapy of the human subject; resecting the carcinoma tumor of the lung to obtain a lung cancer sample; obtaining expression information for the seven gene signature in the lung cancer sample obtained from said subject: normalizing the expression information to generate normalized expression information; determining a benefit score based on the normalized expression information; comparing the benefit score to a pre-determined risk threshold; classifying a human subject as having “high risk” of tumor progression and as more likely to benefit from an aggressive therapy based on the comparison between the benefit score and the pre-determined benefit threshold; and treating the human subject with an aggressive therapy in response to determining the human subject has a “high benefit” of tumor response to chemotherapy. The seven gene signature can include RRM2, AURKA, NKX2-1, COL4A3, ATP8A1, C1orf116, and HSD17B6. A set of 7 genes that are predictive of response to chemotherapy of the human subject can further be identified; a regression coefficient for each gene included in the set of 7 genes can further be calculated; a regression value threshold using a cross validation process can further be determined; and the seven gene signature from the set of 7 genes based on a comparison of the regression coefficient for each gene to the regression value threshold can be identified. The normalized expression information can adjust the expression information for technical assay variation, background noise, and RNA content. A validation dataset including expression information obtained from a plurality of fresh frozen or formalin-fixed paraffin-embedded lung cancer tumor samples can be generated; the benefit score can be calculated through a combination of three principle components; the contribution for each gene included in the seven gene signature to each principle component can be determined; the three principle components can be combined to generate a one-dimensional benefit score; a benefit score can be determined for each patient based on the expression of the seven genes in the 7-gene signature; a benefit threshold can be determined from the training dataset; and each specific patient can be classified as either in the high- or low-benefit group by comparing the benefit score calculated from the seven gene signature to the benefit threshold.

Another embodiment provides a method of predicting the tumor progression of a subject diagnosed with lung cancer comprising determining expression levels of the following genes: RRM2, AURKA, NKX2-1, COL4A3, ATP8A1, C1orf116, and HSD17B6, in a lung cancer sample obtained from the subject, wherein an alteration in the expression of the genes, as compared to an expression threshold in lung cancer, indicates that the subject will have a ‘high risk’ of tumor progression.

The subject can further be treated with one or more adjuvant chemotherapies. The lung cancer sample can be a fresh frozen tumor (FF) sample. The lung cancer sample can be a formalin-fixed paraffin-embedded (FFPE) tumor sample. The lung cancer can be non-small cell lung cancer (NSCLC), lung adenocarcinoma (ADC), or lung squamous cell carcinoma (SCC). The one or more adjuvant chemotherapies can be vinorelbine, cisplatin, carboplatin, gemcitabine, paclitaxel, topotecan, docetaxel, irinotecan, pemetrexed, etoposide, or any combination thereof. Administering an adjuvant chemotherapy (ACT) to a subject predicted to benefit from an ACT can improve survival of the subject.

### BRIEF DESCRIPTION OF THE DRAWINGS

The accompanying drawings are included to provide a further understanding of the methods and compositions of the disclosure, are incorporated in, and constitute a part of this specification. The drawings illustrate one or more embodiments of the disclosure, and together with the description serve to explain the concepts and operation of the disclosure.

FIG. 1 illustrates an exemplary process 100 for predicting patent response to adjuvant chemotherapy (ACT).

FIG. 2 illustrates an exemplary machine learning system 220 that may be used to identify a gene signature and generate benefit and/or risk scores based on expression data obtained for the genes included in the gene signature.

FIG. 3 illustrates an exemplary cross validation curve having a regression value threshold of 0.8.

FIGS. 4A-4B show predictive performance of the signature in an independent validation dataset including 166 patients, including 49 patients treated with an adjuvant therapy and 127 patients receiving no ACT. FIG. 4A shows a curve illustrating the overall survival of ACT-treated and non-ACT-treated patients in the predicted benefit group (n=88).

FIG. 4B shows a curve illustrating the overall survival of ACT-treated and non-ACT-treated patients in the predicted non-benefit group (n=88).

FIGS. 5A-5B show predictive performance of the signature in another independent validation dataset including 90 patients including 49 patients treated with vinorelbine+cisplatin, and 41 patients receiving no ACT. FIG. 5A shows a curve illustrating the overall survival of ACT-treated and non-ACT-treated patients in the predicted benefit group (n=45). FIG. 5B shows a curve illustrating the overall survival of ACT-treated and non-ACT-treated patients in the predicted non-benefit group (n=45).

FIGS. 6A-6B show predictive performance of the signature in an independent dataset with gene expression measured from FFPE tissue samples in 207 patients. FIG. 6A shows a curve illustrating the overall survival of ACT-treated and non-ACT-treated patients in the predicted benefit group. FIG. 6B shows a curve illustrating the overall survival of ACT-treated and non-ACT-treated patients in the predicted non-benefit group.

FIGS. 7A-7D show prognostic performance of the signature in four independent datasets with 62, 90, 117 and 133 lung ADC patients, respectively, measured from fresh frozen tumor samples. FIG. 7A shows a curve illustrating the overall survival of high risk and low risk patients among the 62 patients with lung ADC from GSE8894 dataset.

FIG. 7B shows a curve illustrating the overall survival of high risk and low risk patients among the 90 patients with ADC from GSE11969 dataset. FIG. 7C shows a curve illustrating the overall survival of high risk and low risk patients among the 117 patients with ADC from GSE13213 dataset. FIG. 7D shows a curve illustrating the overall survival of high risk and low risk patients among the 133 patients with ADC from UT Lung SPORE dataset.

FIG. 8 shows prognostic performance of the signature in a dataset with gene expression measured from FFPE tissue samples in 166 lung ADC patients without ACT.

FIG. 9 is a block diagram illustrating an example computer system that may be used to implement the machine learning system of FIG. 2 and the algorithms used to perform the processing steps disclosed in FIG. 1.

### DETAILED DESCRIPTION

The present disclosure provides a method of predicting the survival of a human subject diagnosed with lung cancer based on the development of a 7-gene signature, and methods of predicting a response to an adjuvant chemotherapy (ACT) using the signature.

### Overview

Different approaches can be used to identify potentially predictive biomarker signatures. First, predictive signatures can be identified based on biologic or functional knowledge and molecular mechanisms. For example, the identification of ERCC1 as a predictive marker for chemotherapy response was based on the molecular mechanisms of chemotherapy and DNA repair pathways. Second, predictive signatures can also potentially be identified by preclinical experiments, such as using human tumor cell lines and xenograft models that show different responses to chemotherapy to generate biomarker signatures associated with these different preclinical therapy response phenotypes. Third, a prognostic signature may also have predictive value. Fourth, predictive signatures can be identified based on genome-wide testing of which genes have interactions between expression level and treatment. This is the most direct way to identify predictive signatures but requires a large cohort of samples with detailed treatment information, clinical outcomes and frozen tissues. Therefore, this approach has been rarely implemented in practice. In the present disclosure, a 7-gene set was identified through the combination of biological and function knowledge together with the prognosis signature.

Multiple studies have been conducted to identify clinical factors that are associated with chemotherapy responses in NSCLC. Cancer and Leukemia Group B (CALGB) recently demonstrated that there is no significant survival benefit of ACT in stage IB NSCLC patients based on a randomized trial (p-value 0.12), while a statistically significant survival advantage was observed for stage IB patients with tumors cm. The Lung Adjuvant Cisplatin Evaluation (LACE) study showed that the chemotherapy effect was higher in patients with better performance status, and there was no interaction between the chemotherapy effect and gender, age, histology, type of surgery, planned radiotherapy, or planned total dose of cisplatin. Currently, a patient's TNM stage is the main clinical variable that provides prognostic information to suggest which patients need ACT. However, the TNM information (or the specific tumor histopathologic subtype) does not predict which patients within a TNM-stage category will derive survival benefit from ACT.

A major goal of “personalized” cancer therapy is to develop tools to sample a patient's tumor, perform molecular analyses, predict the patient's prognosis, and select the best treatment. Recent developments in genome-wide expression profiling of human tumors have shown that histologically similar tumors can exhibit different gene or protein expression profiles, and these tumor profiles are associated with different patient prognoses. The ultimate clinical use of these signatures, in practice, would be to profile tumors and then determine the best treatment strategy for each individual patient. Although many prognosis signatures were reported in lung cancer, few predictive signatures for ACT response, which would have a direct impact on clinical decisions for early-stage NSCLC patients, are available. All prediction markers for chemotherapy response in lung cancer are still in the research stage, and no commercialized clinical assays are available yet.

In the present disclosure, a clinical assay, based on Nanostring nCounter platform coupled with a risk stratification algorithm to predict patient response to adjuvant chemotherapy (ACT) after tumor resection was developed and validated. This clinical-grade assay can measure mRNA expression levels from fresh frozen (FF) and archived formalin-fixed paraffin-embedded (FFPE) tumor samples and can assist clinicians in the clinical decision-making for individual non-small-cell lung cancer (NSCLC) patients. Using this gene signature, it was shown that the predicted benefit group showed significant improvement in survival after ACT, while the predicted non-benefit group showed no survival benefit.

Currently, most biomarkers are based on protein expression, which requires high quality antibodies. In this disclosure, a signature based on the mRNA expression of seven genes measured from FF and FFPE tumor samples was developed. The predictive performance of this 7-gene signature was validated in independent datasets. From a patient care prospective, the clinical assay and the predictive algorithm will permit clinicians to identify patients—before the start of therapy—who are most and least likely to benefit from ACT. It will have immense clinical benefit in terms of planning treatments for individual patients. This Clinical Laboratory Improvement Amendments (CLIA)-certifiable clinical assay for FFPE samples with the optimized algorithm (formula) to derive the risk scores—a complete package for a clinical diagnostic assay—can provide a great opportunity for the development of a clinical device for regulatory approval.

Machine Learning System

A machine learning system for generating gene signature and predicting ACT response based on gene signature expression data is provided.

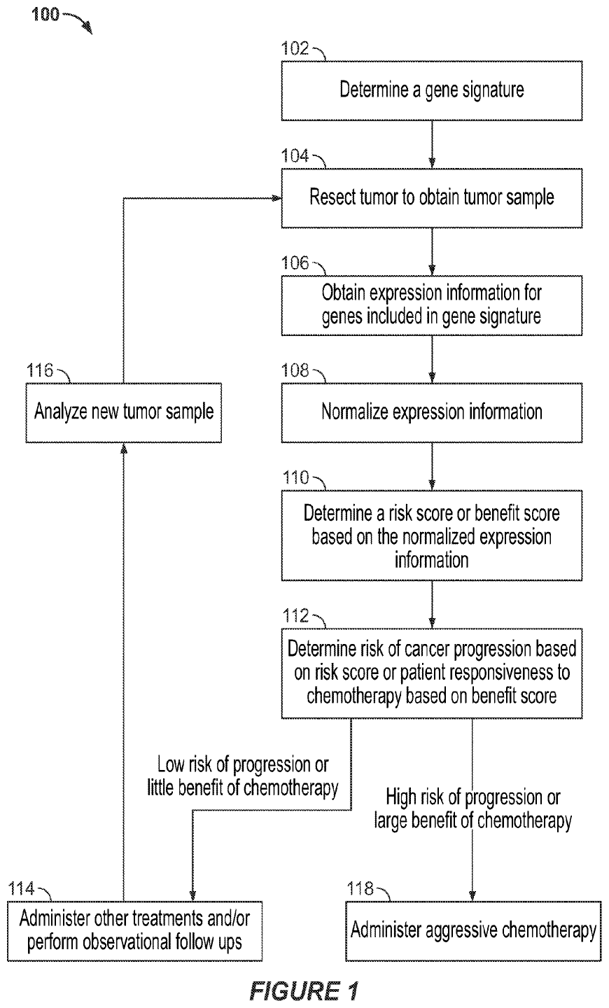

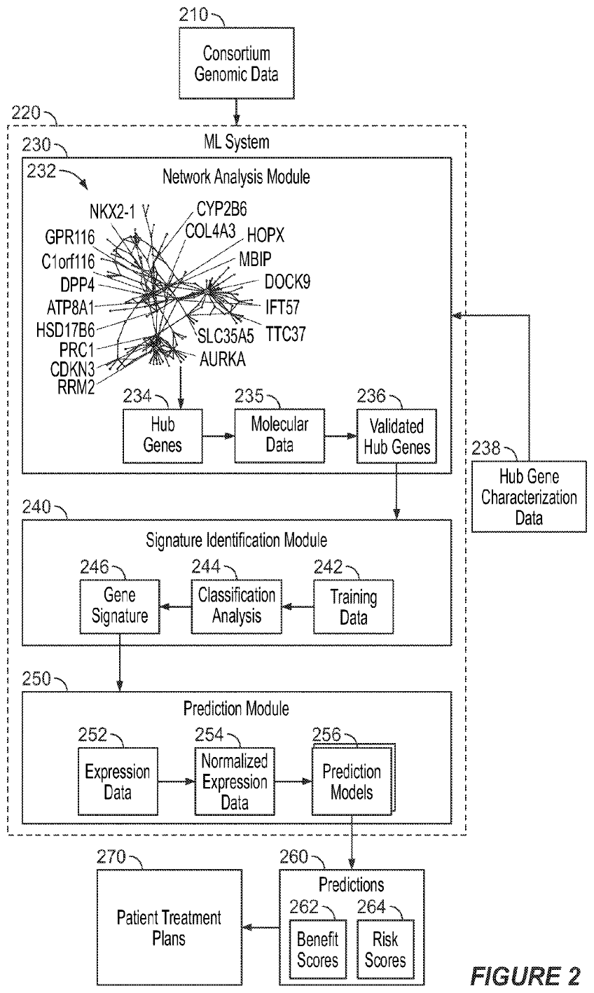

Referring to FIG. 1, which illustrates an exemplary process 100 for predicting patent specific cancer treatment plan. At 102, the method may include determining a gene signature that is predictive of a patent response to ACT and disease prognosis. The gene signature may be determined, for example, by using the machine learning system shown in FIG. 2. For example, a network analysis may be performed to identify a plurality of hub genes included in a gene library constructed from tumor samples of a cohort of human subjects. The hub genes may each be linked to seven or more genes in a gene network (e.g., a cancer survival related gene network, an ACT response related gene network, and the like). Other types of molecular data may be used to further refine hub genes. A training dataset may then be used to identify a gene signature from the hub genes. For example, the training dataset may include expression information for the hub genes obtained from each patient sample and the patient outcome that corresponds to each set of expression information. The gene signature may include any number of genes (e.g., a 12 gene signature, a 7 gene signature, and the like) that are found in patient tissue samples (e.g., patient tumor samples) included in the cohort used to construct the gene network and are known to have expression information that may be used to predict a patient response to one or more chemotherapies (e.g., ACT) and/or a disease prognosis (e.g., tumor progression).

At 104, a tumor sample may be resected from the patient. For example, a tumor may be resected from the lung of a patient to obtain a lung cancer sample. At 106, gene expression information for the genes included in the gene signature may be obtained from the sample. The gene expression information may include a gene expression profile obtained using an mRNA detection technique. The gene expression information may be normalized at 108 to obtain normalized expression information. The normalized expression information may, for example, adjust the expression information for technical assay variation, background noise, and/or RNA content.

At 110, a risk score or a benefit score may be determined based on the normalized expression information. For example, a prediction model may receive the normalized expression information as input and generate a benefit score indicating a patient response to a chemotherapy as output. To predict a patient response to ACT, the benefit score may be compared to a pre-determined benefit threshold. The benefit threshold may be determined based on a training dataset including benefit scores generated from the seven gene expression information for a cohort of patients having known responses to chemotherapies.

In other examples, the prediction model may receive the normalized expression information as input and generate a risk score indicating a patient patent disease prognosis as output. To predict the risk of tumor progression, the risk score may be compared to a pre-determined risk threshold. The risk threshold may be determined based on a training dataset including risk scores generated from the seven gene expression information for a cohort of patients having known disease prognoses (e.g., known rates of tumor progression).

If at 112, the benefit score or the risk score indicates the patient will be responsive to chemotherapy (i.e., the benefit score is above the cut-off score of the pre-determined benefit threshold or the risk score is above the cut-off score of the pre-determined risk threshold), the patient may be treated with a particular chemotherapy treatment at 114. For example, if the benefit score or the risk score indicates the patient is likely to benefit from ACT (i.e., the patient has a high risk of cancer progression and or will be responsive to ACT, the patient may be treated with ACT. If at 112, the benefit score or the risk score indicates the patient has a low risk of cancer progression and or will not be responsive to chemotherapy (i.e., a benefit score that is below the cut-off score of the pre-determined benefit threshold or a risk score that is below the cut-off score of the pre-determined risk threshold), other treatment options such as immunotherapies or targeted therapies may be administered and/or observational follow-ups may be performed by obtaining and analyzing a new tumor sample from the patient at a later date at 116. If at 112, the benefit score or the risk score indicates the patient has a high risk of cancer progression and or will be responsive to chemotherapy (i.e., a benefit score that is above the cut-off score of the pre-determined benefit threshold or a risk score that is above the cut-off score of the pre-determined risk threshold), an aggressive chemotherapy such as adjuvant chemotherapy may be administered to the patient at 118.

The benefit score or the risk score generated by the prediction model may also be a classification prediction. For example, the risk score may classify the patient as in a “low risk” group or “high risk” group and the benefit score may classify the patient as in a “response” group or “non-responsive” group. If at 112 the risk score classifies the patient in the “low risk” group or the benefit score classifies the patient in the “non-responsive” group, the patient may have a low risk of tumor progression and little responsiveness to chemotherapy and treatment other than adjuvant chemotherapy treatment may be administered to the patient at 114. If at 112, the risk score classifies the patient in the “high risk” group or the benefit score classifies the patient in the “responsive” group, the patient may have a high risk of tumor progression and may be responsive to chemotherapy and adjuvant chemotherapy may be administered to the patient at 118.

Referring to FIG. 2, which illustrates an exemplary machine learning system 220 that may be used to identify a gene signature and generate risk scores and benefit scores based on expression data obtained for the genes included in the gene signature. The machine learning system 220. may include a plurality of software modules that provide the functionality of the machine learning system 220. The machine learning system 220 may be implemented on any computing device including a processor and memory.

The machine learning system 220 may receive consortium genomic data 210 including genes identified from tumor samples of a cohort of patients (e.g., 500 patients having the same type of carcinoma or another form of cancer). The consortium genomic data 210 may include a large volume of genes (e.g., hundreds or thousands of genes) known to be associated with a lung cancer outcome. For example, the genes may be associated with an increased survival rate, a particular disease prognosis, a low rate of disease progression, a high rate of disease progression, a positive response to chemotherapy of another treatment, a negative response to a particular treatment, and the like.

A network analysis module 230 may generate a gene network 232 that maps the genes included in the consortium genomic data 210. The gene network 232 may display each gene as a node in the network and associations between genes may be shown as edges that connect the nodes. The most connected genes in the gene network 232 (i.e., the genes corresponding to the nodes connected to the most edges) may be identified as hub genes 234. Other types of molecular data 235, for example, copy number variation, gene function screen data, functional data, and the like may be used to refine hub genes. The predictive ability of the hub genes 234 may be verified using hub gene characterization data 238. For example, the gene hub characterization data 238 may include gene expression data for the hub genes 234 and patient outcomes (i.e., survival, treatment response, etc.) associated with the expression data of each hub gene 234. The hub genes 234 that are validated by the hub gene characterization data 238 are identified as validated hub genes 236 and provided to the signature identification module 240 for further analysis.

The signature identification module 240 may identify more or more gene signatures 246 from the verified hub genes 236. To identify a gene signature 246, the signature identification module 240 may train a prediction model 256 to generate predictions 260 of patient outcomes (e.g., responsiveness to treatment and/or disease progression) based on gene expression data. For example, the signature identification module 240 may train a prediction model 256 (e.g., with three supervised principal components) to classify the predictive ability of the verified hub genes 236 using a training data 242. The training data 242 may include gene expression information for each hub gene 236 and the patient outcomes associated with a particular expression profile for each hub gene 236. The training data 242 may include gene expression information obtained from a cohort of patients and may include expression profiles for hundreds or thousands of patient samples. A portion of the training data 242 may be withheld from training the prediction models 256 and may be used as test data and or validation data.

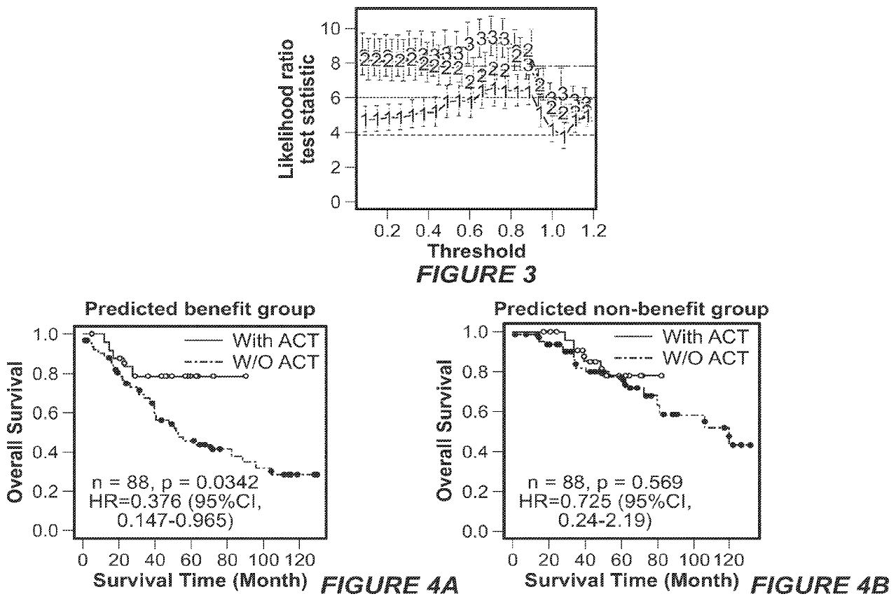

To identify the gene signature 246, a cross validation process may be used to combine the expression from each gene and determine the output of the prediction model 256. For example, cross validation curves illustrating the relative prediction accuracy associated with each gene in the verified hub genes 236 may be generated for gene expression data that was withheld from the training dataset. A classification analysis 244 may then be performed on the cross-validation curves to determine a threshold value that distinguishes the validated hub genes 236 that have the highest relative predictive ability. Now referring to FIG. 3, which illustrates an exemplary cross validation curve used to perform one embodiment of the classification analysis 244. The cross-validation curve shown in FIG. 3 includes curves for the prediction model. The classification analysis 244 performed on the curves identified a threshold value (i.e., a regression value threshold) of 0.8 for the prediction model. The validated hub genes 236 that are determined as predictive based on the classification analysis 244 are included in the gene signature 246. The gene signature 246 is then used by the prediction module 250 to generate predictions 260 that may be used to determine patient treatment plans 270.

The prediction module 250 receives expression data 252 for the genes included in the gene signature 246. The expression data 252 for the gene signature 246 may be obtained from a patient tissue sample (e.g., a patient tissue sample) using RNA detection techniques and or other methods. To make the prediction models 256 more robust, the prediction module 250 may normalize expression data 252 before it is input into the prediction model 256. For example, the normalized expression data 254 may adjust the expression data 252 for technical assay variation, background noise, and/or RNA content. The normalized expression data 254 is then received by the prediction model 256. The prediction module 250 may include multiple prediction models 256 and each model may generate its own prediction based on the normalized expression data 254. For example, the prediction module 250 may include three principle components that may each generate a score for every gene included in the gene signature 246. The prediction module 250 may aggregate the scores for each gene included in the gene signature 246 and each principal component in the prediction model 256 to generate a prediction 260 for a particular patient and patient outcome. For example, a prognosis prediction model may generate risk scores for each gene included in the gene signature 246 and each principle components (e.g., 21 total scores for a 7 gene signature) and aggregate the 21 risk scores into an aggregate risk score that is used to determine a disease prognosis for a patient. The risk score 260 may include, for example, a predicted patient survival rate or a response to a particular treatment. In other examples, a treatment response prediction model may generate benefit scores for each gene included in the gene signature 246 and each principle components (e.g., 21 total scores for a 7 gene signature) and aggregate the 21 benefit scores into an aggregate benefit score that is used to determine a disease prognosis for a patient. The benefit score 260 may include, for example, a predicted patient response to a particular treatment. Healthcare providers may use the benefit scores and the risk scores 260 to generate patient treatment plans 270 including chemotherapy and other treatments that are administered to patients.

Methods of Use

The machine learning system can be used to predict the survival of a subject diagnosed with lung cancer, and to predict a response to an ACT.

A method of predicting the response to chemotherapy of a human subject diagnosed with lung cancer comprising; determining a seven gene signature that predicts the response to chemotherapy of the human subject; resecting the carcinoma tumor of the lung to obtain a lung cancer sample; obtaining expression information for the seven gene signature in the lung cancer sample obtained from said subject: normalizing the expression information to generate normalized expression information; determining a benefit score based on the normalized expression information; comparing the benefit score to a survival threshold; determining, the human subject is likely to respond to chemotherapy based on the comparing the benefit score to the benefit threshold is provided.

A set of 7 genes that are predictive of the response to chemotherapy of the human subject can further be identified; a regression coefficient for each gene included in the set of 7 genes can further be calculated; a regression value threshold using a cross validation process can further be determined; and the seven gene signature from the set of 7 genes based on a comparison of the regression coefficient for each gene to the regression value threshold can be identified. The normalized expression information can adjust the expression information for technical assay variation, background noise, and RNA content. A validation dataset including expression information obtained from a plurality of fresh frozen or FFPE lung cancer samples can further be generated; a benefit score of a subject can be determined by combining the expression information for each gene included in the seven gene signature; and a benefit group of a subject can be determined by comparing the subject benefit score calculated from the seven gene signature to a predetermined benefit threshold.

A method of predicting the tumor progression in a subject diagnosed with lung cancer comprising determining expression levels of the following genes: RRM2, AURKA, NKX2-1, COL4A3, ATP8A1, C1orf116, and HSD17B6, in a lung cancer sample obtained from the subject, wherein an alteration in the expression of the genes, as compared to the predetermined threshold in lung cancer, indicates that the subject will have ‘high risk’ of tumor progression.

Lung Cancer

Lung cancer can include any cancer that arises from the lung. There are 2 main types of lung cancer: non-small cell lung cancer (NSCLC) and small cell lung cancer (SCLC). About 80% to 85% of lung cancers are NSCLC. The main subtypes of NSCLC are adenocarcinoma (ADC), squamous cell carcinoma (SCC), and large cell carcinoma. These subtypes, which start from different types of lung cells are grouped together as NSCLC because their treatment and prognoses (outlook) are often similar. ADC start in the cells that would normally secrete substances such as mucus. This type of lung cancer occurs mainly in current or former smokers, but it is also the most common type of lung cancer seen in non-smokers. It is more common in women than in men, and it is more likely to occur in younger people than other types of lung cancer. ADC is usually found in the outer parts of the lung and is more likely to be found before it has spread. Patients with in situ ADC tend to have a better outlook than those with other types of lung cancer. SCC starts in squamous cells, which are flat cells that line the inside of the airways in the lungs. They are often linked to a history of smoking and tend to be found in the central part of the lungs, near a main airway (bronchus). Large cell (undifferentiated) carcinoma can appear in any part of the lung. It tends to grow and spread quickly, which can make it harder to treat. About 10% to 15% of all lung cancers are SCLC. This type of lung cancer tends to grow and spread faster than NSCLC. About 70% of people with SCLC will have cancer that has already spread at the time they are diagnosed. Since this cancer grows quickly, it tends to respond well to chemotherapy and radiation therapy. Unfortunately, for most people, the cancer will return at some point.

In one embodiment, the lung cancer can be non-small cell lung cancer (NSCLC), lung adenocarcinoma (ADC), or lung squamous cell carcinoma (SCC).

The lung cancer can be an early stage lung cancer, or a late stage cancer. For example, the lung cancer can be stage 1, when the cancer is found in the lung, but it has not spread outside the lung; stage 2, when the cancer is found in the lung and nearby lymph nodes stage 3, when the cancer is in the lung and lymph nodes in the middle of the chest; stage 3A, when the cancer is found in lymph nodes, but only on the same side of the chest where cancer first started growing; stage 3B, when the cancer has spread to lymph nodes on the opposite side of the chest or to lymph nodes above the collarbone; or stage 4, when the cancer has spread to both lungs, into the area around the lungs, or to distant organs.

The lung cancer sample can be collected from any lung cancer subtypes, at any stage. For example, the lung cancer sample can be collected from a stage 1, 2, 3, 3A, 3B or 4 NSCLC, from a stage 1, 2, 3, 3A, 3B or 4 ADC, or from a stage 1, 2, 3, 3A, 3B or 4 SCC. The sample can be collected from the primary tumor (i.e., the initial cancerous lesion), from a secondary tumor (i.e., any cancerous lesion subsequently discovered in the lung), from a lymph node, or from a distant metastatic lesion (i.e., a metastasis of the primary or secondary lung tumor, localized outside of the lung tissue). Several samples collected from a single patient can also be analyzed at one. For example, a patient diagnosed with a primary tumor localized in the lung, that has spread to lymph nodes and to a distant organ can underwent surgery to remove the lung lesion, the affected lymph modes, and the distant lesion. The benefit score described herein can be run on one of the samples (i.e., on the primary lesion, on the lymph node, or on the distant lesion), or be run on more than one of the samples collected, to assess if different lesions may respond differently.

The methods described herein can further comprise treating the subject with one or more adjuvant chemotherapies Lung cancer treatment options can include surgery, adjuvant chemotherapy, radiotherapy, targeted therapy, immunotherapy or a combination thereof. As used herein, an “adjuvant chemotherapy” or “ACT” refers to any chemotherapy that is administered to a patient in addition to and after an initial or primary treatment that removes all detectable disease, to maximize its effectiveness. An adjuvant treatment, or ACT can then refer to any additional treatment that is administered to a patient after an initial treatment where a risk of relapse remains due to the presence of undetected disease. In patients with lung cancer, the primary treatment is usually surgery to remove cancerous cells; therefore, the ACT can be administered after the patient underwent surgery. In such an example, the ACT aims at reducing the chances of recurrence by eliminating cancer cells left after the surgery. The ACT can be administered to a patient with lung cancer independently of the type of primary treatment that was initially administered to the patient.

The most common chemotherapy used to treat lung cancer can include cisplatin, carboplatin, paclitaxel, albumin-bound paclitaxel such as nab-paclitaxel, docetaxel, gemcitabine, vinorelbine, etoposide, pemetrexed, topotecan, irinotecan, or combination thereof. The efficacy and outcome observed with various ACTs are similar, with the choice of one or the other ACT being based on health status of the patient, tumor types, patient's age or a combination thereof. The risk score described herein allows for the prediction of the response to a patient to an ACT. Lung cancer ACTs, which include ACTs that are most commonly used in patient with lung cancer, and which include chemotherapeutic agents such as cisplatin, carboplatin, paclitaxel, albumin-bound paclitaxel such as nab-paclitaxel, docetaxel, gemcitabine, vinorelbine, etoposide, pemetrexed, topotecan, and irinotecan have a similar mechanism of action (they interfere with DNA replication, therefore targeting and killing the fastest proliferating cells, such as cancerous cells). An ACT can therefore be a drug that interferes or inhibits DNA replication of cancerous cells. The response to any other ACT having a similar mechanism of action is anticipated to be predictable using the presently presented risk score; therefore, any other ACT having a similar mechanism of action can be used. The 7-gene signature described herein has been developed using data of patients who underwent surgery and were treated with a wide range of chemotherapy; and was proven robust to predict patient response to various types of ACT. Therefore, the 7-gene signature described herein can be used to predict the response to any adjuvant chemotherapy.

In one embodiment, the one or more adjuvant chemotherapies can be vinorelbine, cisplatin, carboplatin, gemcitabine, paclitaxel, topotecan, docetaxel, irinotecan, pemetrexed, etoposide, or any combination thereof.

The benefit score described herein can be used to predict the likelihood of a subject to respond to ACT (i.e., to evaluate the likelihood of a subject to reply to chemotherapy, as the first drug received after surgery). However, it is understood that the benefit score can be used to assess such likelihood at any point during the progression of the disease. For example, a lung cancer sample can be collected during the first surgery, after lung cancer diagnostic, and prior to any additional treatment beside surgery. The lung cancer sample can also be collected in a patient that already underwent surgery and one or more round of an additional therapy. For example, a patient can undergo surgery, and be administered an additional therapy, such as radiation therapy, or immunotherapy. A patient with a recurrent lung cancer, that has not been treated with chemotherapy can still be responsive to chemotherapy, even after failure of other therapies. A risk score as described herein can therefore be assessed, to evaluate the likelihood of a chemotherapeutic treatment to increase survival in the patient, and the likelihood to respond to the chemotherapy.

The ACT can be administered to a patient. The terms “administration of” and or “administering” should be understood to mean providing an ACT in a therapeutically effective amount to the patient in need of treatment. Administration routes can be enteral, topical or parenteral. As such, administration routes include but are not limited to intracutaneous, subcutaneous, intravenous, intraperitoneal, intraarterial, intrathecal, intracardiac, intradermal, transdermal, oral, sublingual buccal, nasal, ocular administrations, as well infusion, inhalation, and nebulization.

The term “subject” as used herein refers to any individual or patient to which the subject methods are performed. Generally, the subject is human, although as will be appreciated by those in the art, the subject may be an animal. In an embodiment, the subject in a patient diagnosed with lung cancer.

In some aspects, administering an ACT to the subject can improve survival of the subject.

Gene Expression Levels

A 7-gene signature described herein can be used to predict a response to a treatment. For example, and as discussed above, a benefit score can be evaluated for a patient, based on the expression levels of the genes in the signature, and the value of the benefit score, when compared to a benefit threshold can indicate the likelihood of the patient to respond to an adjuvant chemotherapy, and the likelihood of the patient to have an increase survival.

In an embodiment, methods described herein can comprise further determining a benefit score of the subject by combining expression information of the genes and predicting a benefit group of the subject by comparing the benefit score of the subject to a predetermined risk threshold.

For example, if a risk score is less than a threshold risk (i.e., if a risk score is lower than a predetermined risk score), the patient can be classified as “low risk” of tumor progression and less likely to benefit from the ACT. A patient classified as “high risk” is more likely to have tumor progression, more likely to have poor survival outcome and more likely to benefit from an ACT, and therefore should be treated with an ACT, as the risk score can indicate a higher likelihood of having an increased survival as a result from the ACT, as compared to a predicted survival in the absence of a treatment with an ACT.

If a benefit score is greater than a threshold risk (i.e., if a benefit score is greater than a pre-defined threshold that has been evaluated), the patient can be classified as “responsive” to chemotherapy and more likely to benefit from the ACT. A patient classified as “non-responsive” to chemotherapy can be more likely to not respond to an ACT such as a drug that interferes or inhibits DNA replication in cancerous cells; therefore, other therapy, such as radiation therapy or immunotherapy can be considered as opposed to ACT that interferes or inhibits DNA replication in cancerous cells, as the lower risk score can indicate a lower likelihood of benefiting the ACT. Additionally, the low likelihood of benefiting from ACT can be associated with a low likelihood of having an increased survival as a result from the ACT, as compared to a predicted survival in the absence of a treatment with an ACT. In such case, another benefit score can be determined at a later time, after an alternative round of therapy that does not include an ACT, is necessary, to re-evaluate the likelihood to respond to an ACT (as therapy, or tumor heterogeneity can result in secondary and metastatic lesions to have different gene expression profile as compared to an initial lesion).

The expression levels of 7 genes in the signature can be determined in various ways. A gene refers to a nucleic acid molecule that encodes a protein. As used herein, the term “nucleic acid molecule” or” oligonucleotide” can refers to polynucleotides such as deoxyribonucleic acid (DNA) or ribonucleic acid (RNA). A nucleic acid molecule can be isolated. The term “isolated nucleic acid molecule” means, that the nucleic acid molecule (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A nucleic acid molecule might be employed for introduction into, i.e. transfection of, cells, in particular, in the form of RNA which can be prepared by in vitro transcription from a DNA template. The RNA can moreover be modified before application by stabilizing sequences, capping, and polyadenylation.

The extracted nucleic acid molecule can be amplified by one of the alternative methods for amplification well known in the art, which include for example: the Xmap® technology of Luminex that allows the simultaneous analysis of up to 500 bioassays through the reading of biological test on the surface of microscopic polystyrene bead; the multiplex PCR that allows the simultaneous amplification of several DNA molecules; the multiplex ligation-dependent probe amplification (MLPA) for the amplification of multiple targets using a single pair of primers; the quantitative PCR (qPCR), which measures and quantify the amplification in real time; the ligation chain reaction (LCR) that uses primers covering the entire sequence to amplify, thereby preventing the amplification of sequences with a mutation; the rolling circle amplification (RCA), wherein the two ends of the sequences are joined by a ligase prior to the amplification of the circular DNA; the helicase dependent amplification (HDA) which relies on a helicase for the separation of the double stranded DNA; the loop mediated isothermal amplification (LAMP) which employs a DNA polymerase with high strand displacement activity; the nucleic acid sequence based amplification, specifically designed for RNA targets; the strand displacement amplification (SDA) which relies on a strand-displacing DNA polymerase, to initiate replication at nicks created by a strand-limited restriction endonuclease or nicking enzyme at a site contained in a primer; and the multiple displacement amplification (MDA), based on the use of the highly processive and strand displacing DNA polymerase from the bacteriophage Ø29. Amplification methods as used herein have been used and tested, and are well known in the art.

As used herein “amplified DNA” or “PCR product” refers to an amplified fragment of DNA of defined size. Various techniques are available and well known in the art to detect PCR products. PCR product detection methods include, but are not restricted to, gel electrophoresis using agarose or polyacrylamide gel and adding ethidium bromide staining (a DNA intercalant), labeled probes (radioactive or non-radioactive labels, southern blotting), labeled deoxyribonucleotides (for the direct incorporation of radioactive or non-radioactive labels) or silver staining for the direct visualization of the amplified PCR products; restriction endonuclease digestion, that relies agarose or polyacrylamide gel or High-performance liquid chromatography (HPLC); dot blots, using the hybridization of the amplified DNA on specific labeled probes (radioactive or non-radioactive labels); high-pressure liquid chromatography using ultraviolet detection; electro-chemiluminescence coupled with voltage-initiated chemical reaction/photon detection; and direct sequencing using radioactive or fluorescently labeled deoxyribonucleotides for the determination of the precise order of nucleotides with a DNA fragment of interest, oligo ligation assay (OLA), PCR, qPCR, DNA sequencing, fluorescence, gel electrophoresis, magnetic beads, allele specific primer extension (ASPE) and/or direct hybridization.

A 7-gene signature described herein was developed using NanoString nCounter platform. NanoString nCounter is a CLIA-certifiable assay, which means the 7-gene signature is ready to be translated into clinical application. The 7-gene signature does not depend on the platform on which the gene expression is assessed. The expression level of the genes or proteins of the signature can be evaluated using any platform suitable to measure gene or proteins expression.

7-Gene Signature

The robust algorithm described herein, uses positive controls, negative controls and house-keeping genes to standardize and normalize the measured gene expression for mRNA extracted from FFPE samples. Using the normalized expression value, a risk score or benefit score for each individual patient can be calculated without comparing the calculated benefit score or risk score with those from a cohort of patients. Therefore, a patient can be evaluated independently of any comparison to a cohort, as a benefit score or a risk score calculated for the patient can by itself indicate the likelihood to have an increase survival with a treatment with an ACT.

A 7-gene signature can include RRM2, AURKA, NKX2-1, COL4A3, ATP8A1, C1orf116, and HSD17B6.

Ribonucleoside-diphosphate reductase subunit M2, also known as ribonucleotide reductase small subunit, is an enzyme that in humans is encoded by the RRM2 gene (access number NM_001034.4) This reductase catalyzes the formation of deoxyribonucleotides from ribonucleotides. Synthesis of the encoded protein (M2) is regulated in a cell-cycle dependent fashion. Transcription from this gene can initiate from alternative promoters, which results in two isoforms that differ in the lengths of their N-termini.

Aurora kinase A also known as serine/threonine-protein kinase 6 is an enzyme that in humans is encoded by the AURKA gene (access numbers NM_001323303.2, NM_001323304.2, NM_001323305.2, NM_003600.4, NM_198433.3, NM_198434.3, NM_198435.3, NM_198436.3, and NM_198437.3). Aurora A is a member of a family of mitotic serine/threonine kinases implicated with important processes during mitosis and meiosis whose proper function is integral for healthy cell proliferation. Aurora A is activated by one or more phosphorylations and its activity peaks during the G2 phase to M phase transition in the cell cycle.

NK2 homeobox 1 (NKX2-1), also known as thyroid transcription factor 1 (TTF-1), is a protein which in humans is encoded by the NKX2-1 gene (access number NM_001079668.3). Thyroid transcription factor-1 (TTF-1) is a protein that regulates transcription of genes specific for the thyroid, lung, and diencephalon. It is also known as thyroid specific enhancer binding protein. It is used in anatomic pathology as a marker to determine if a tumor arises from the lung or thyroid.

Collagen alpha 3(IV) chain is a protein that in humans is encoded by the COL4A3 gene (access number NM_000091.5). Type IV collagen, the major structural component of basement membranes, is a multimeric protein composed of 3 alpha subunits. These subunits are encoded by 6 different genes, alpha 1 through alpha 6, each of which can form a triple helix structure with 2 other subunits to form type IV collagen.

ATPase phospholipid transporting 8A1 is a protein that in human is encoded by the ATP8A1 gene (access numbers NM_001105529.1, NM_006095.2). The P-type adenosine triphosphatases (P-type ATPases) are a family of proteins which use the free energy of ATP hydrolysis to drive uphill transport of ions across membranes. Several subfamilies of P-type ATPases have been identified. One subfamily catalyzes transport of heavy metal ions. Another subfamily transports non-heavy metal ions (NMHI). The protein encoded by this gene is a member of the third subfamily of P-type ATPases and acts to transport amphipaths, such as phosphatidylserine.

C1orf116, also known as SARG is an open reading frame located in the chromosome 1 (access numbers NM_001083924.2, NM_023938.6).

Hydroxysteroid 17-beta dehydrogenase 6 is an enzyme that in humans is encoded by the HSD17B6 gene (access number NM_003725.4). The protein encoded by this gene has both oxidoreductase and epimerase activities and is involved in androgen catabolism. The oxidoreductase activity can convert 3 alpha-adiol to dihydrotestosterone, while the epimerase activity can convert androsterone to epi-androsterone. Both reactions use NAD+ as the preferred cofactor. This gene is a member of the retinol dehydrogenase family.

Tumor Sample

Nucleic acid molecules may be extracted from a sample, by any method known in the art including by using organic solvents such as a mixture of phenol and chloroform, followed by precipitation with ethanol. Among other methods of extracting cell-free nucleic acid, one such method includes, for example, using polylysine-coated silica particles. Alternatively, the cell-free DNA may be extracted using commercially available kit such as, for example, QIAamp® DNA minikit (Qiagen, Germantown, MD). The term “sample” may include tumor sample, such as biopsies, tumor sample collected during surgery and the like. In various embodiments, the mean use to collect the sample may contain a preservative. The preservative may include preservatives as hydrochloric acid, boric acid, acetic acid, toluene or thymol. In some embodiments, the upon collection the sample can be fixed at ultra-low temperature or using a fixative agent such as formalin.

To translate a molecular cancer biomarker into a clinical device, a biomarker must be developed, optimized, and validated using a platform suited to the analysis of routine clinical formalin-fixed paraffin-embedded (FFPE) specimens. Development of reliable clinical assays from FFPE tumor samples together with an accurate predictive algorithm for response to chemotherapy have an immediate impact on cancer patient care. However, due to the degradation and chemical alteration in RNAs extracted from FFPE samples, measuring the mRNA expression from FFPE samples is a major challenge. To address this challenge, the 7-gene signature was optimized, and the experimental procedures performed to create a new algorithm to calculate risk scores and assign risk groups for individual patients based on FFPE samples.

In one embodiment, the lung cancer sample can be a fresh frozen tumor (FF) sample. In another embodiment, the lung cancer sample can be a formalin-fixed paraffin-embedded (FFPE) tumor sample.

The compositions and methods are more particularly described below, and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### EXAMPLES

### Example 1

### Identification of a 7-Gene Signature

A 7 gene signature has been identified from a selection of 18 hub genes. To determine the 18-hub gene set, 797 genes were identified in the Director's Challenge Consortium dataset whose expression levels were associated with patients' overall survival time (FDR<10%). Next, constructed a lung cancer survival-related gene network was constructed based on expression changes of these 797 genes across 442 lung cancer samples in the Consortium dataset. To construct the network, the association between the expression level of each probeset and survival time was evaluated using multivariate Cox model adjusted for age, cancer stage, and sample processing sites. The false discovery rate (FDR) was calculated from a beta-uniform mixture model (Pounds S, Morris SW. Estimating the occurrence of false positives and false negatives in microarray studies by approximating and partitioning the empirical distribution of p-values. Bioinformatics 2003; 19:1236-42 35). All probesets that passed the FDR criteria (FDR<10%) were included in gene network analysis. When there are multiple probesets corresponding to a single gene, the expression levels from the probesets were averaged to derive the gene level expression. The Sparse PArtial Correlation Estimation (SPACE) algorithm (Peng et al., 2009) was used to construct the network of survival-associated genes using their expression values in the Consortium dataset. From the constructed gene network, genes with at least 7 connections to other genes were identified as “hub” genes.

From the network, 18 hub genes were identified that are connected with at least 7 other genes. Among these 18 genes, RRM2, AURKA, PRC1, and CDKN3 are associated with poor prognosis, while the remaining 14 genes are associated with good prognosis.

A 12 gene signature was then identified from the 18 hub genes. To derive the 12 gene signature, 7 out of the 18 hub genes were found to have significant genetic aberration in lung cancer using the Tumorscape program (world-wide-web at broadinstitute.org/tumorscape), including a key lung cancer driver gene (NKX2-1) (Weir et al., 2007). Furthermore, 9 out of the 18 hub genes were “synthetic lethal” with paclitaxel for NSCLC (i.e., siRNA gene-specific knockdowns which killed NSCLC cells only in the presence of paclitaxel). See, e.g., Whitehurst et al., 2007. In total, 12 out of 18 hub genes either have genetic aberration or are ‘synthetic lethal’ for paclitaxel in lung cancer. These genes are DOCK9, RRM2, AURKA, HOPX, NKX2-1, TTC37, COL4A3, IFT57, Clorf116, HSD17B6, MBIP, and ATP8A1. Because these 12 genes are “hubs” of the survival related genes and play roles in cell response to chemotherapy drugs or have genetic aberrations in lung cancer, each gene included in the 12-gene set may have some capacity to predict survival benefits from ACT in NSCLC.

To improve the efficiency and reduce the cost of performing a personalized patient response analysis in clinical settings, a 7-gene signature was identified. In various embodiments, the 7-gene signature may be identified from a set of 12 or more genes that predicts the likelihood a patient will benefit (i.e., improve the patient's survival) from a particular cancer treatment (e.g., ACT). To derive the 7 gene signature from the 12 gene set a supervised principal component analysis was performed. The supervised principal component analysis is similar to a traditional principal component analysis (PCA), except that it has two additional steps before conducting a traditional PCA: first, compute (univariate) Cox regression coefficients for each gene; and second, form a reduced data matrix containing of only those genes whose univariate coefficient exceeds a threshold for the regression coefficient (θ) in absolute value. For example, (Tang et al. 2013) set θ=0.2 and the supervised principal component analysis model selected 11 out of the 12 genes (DOCK9 is excluded). To identify the 7 gene signature from the 12 gene set, a cross-validation process may be used to determine the value of the threshold for the regresssion coefficent (θ). For example, a cross validation of the log partial likelihood ratio statistic may be used to determine the best value of the threshold for the regression coefficient (θ). (Bair et al. 2006). FIG. 3 shows the cross-validation curves for the first three principal components. Each model is trained, and then the log partial likelihood ratio test (LRT) statistic is computed on the left-out data. All of the three principal components are significant for θ∈(0.7, 0.8). Therefore, the best threshold for the regression coefficient was determined to be around 0.8. Seven out of the twelve genes in the original gene signature have univariate Cox regression coefficients greater than the threshold of 0.8 (RRM2, AURKA, NKX2-1, COL4A3, ATP8A1, C1orf116 and HSD17B6). These seven genes were selected as the seven gene signature based on the comparison of the regression coefficient for each gene to the regression coefficient threshold.

### Example 2

### Generating a Benefit/Risk Score Using the Seven Gene Signature

The prognostic performance of the supervised principal component analysis model was validated using four fresh frozen datasets: GSE8894, GSE11969, GSE13213 and UT Lung SPORE dataset, as well as a dataset from the MD Anderson Cancer Center using FFPE samples. The predictive performance of the supervised principal component analysis model was validated using the fresh frozen UT Lung SPORE and GSE14814 data as well as the MD Anderson Cancer Center FFPE data. The supervised principal component analysis model was trained and supervised with a threshold of 0.8 on the Consortium data and the first three principal components are used to calculate the risk score. Tang et al. (2013). To generate the risks scores a traditional PCA was performed on each gene included in the seven gene signature. Let X, a 440×7 matrix, denote the centered expression data of the seven genes for all 440 patients in the consortium dataset. PCA was considered as a transformation with projection matrix W such that F=XW, which maps the data X from the original space of 7 dimensions to a new space of p dimensions. Only the first three principal components were selected, p=3. The truncated transformation is in the form of Fₜ=XWₜ, where Fₜ is a 440×3 matrix, W t is a 7×3 projection matrix. For a new expression data matrix X, (test data, i.e., fresh frozen UT Lung SPORE, GSE14814 data and the MDACC FFPE data), the first three principal components scores are formed by \hat{F}ₜ=\tilde{X}ₙŴₜ, where \tilde{X}ₙ is Xₙ centered by the column means of X and Ŵₜ is calculated from the training data,

$${\hat{W}}_{t} = [ \begin{matrix}- 0.3756 & - 0.284 & 0.2174 \\ - 0.292 & - 0.3934 & 0.2971 \\ 0.4071 & - 0.7192 & - 0.4678 \\ 0.379 & - 0.03 & 0.6707 \\ 0.3267 & - 0.3531 & 0.4029 \\ 0.4416 & 0.1705 & - 0.1527 \\ 0.4032 & 0.3043 & 0.1004\end{matrix} ] .$$

To combine the first three principal components scores for each gene in the seven gene signature into three one-dimensional risk scores for each principal component, the inventors performed the aggregation method below. The three one-dimensional risk scores reflect the expression information for all genes included in the seven gene signature. To perform the aggregation method, first, the training expression data covariance matrix have Eigen decomposition in the form of X′X=UDU′, where U is a square matrix whose i-th column represents the i-th eigenvector and D is a diagonal matrix whose diagonal elements, dᵢ's, are the corresponding eigenvalues in descending order. Let Ũₜ be the first three columns of U with each column scaled by square root of the corresponding three eigenvalues in D, i.e., (\sqrt{d₁}, \sqrt{d₂}, \sqrt{d₃})=(58.6075,29.0282,26.7334); let (uₜ₁, uₜ₂, uₜ₃) be the column sums of absolute value of Ũₜ; and \tilde{F}ₜ=\tilde{X}ₙŴₜ as defined previously by the test data. The then method scales \tilde{F}ₜ by calculating

$${\tilde{S}}_{t} = {\tilde{F}}_{t} \cdot J \cdot ( \frac{1}{{u}_{t 1}} , \frac{1}{{u}_{t 2}} , \frac{1}{{u}_{t 3}} ) .$$

Then a Cox regression model is fit on the training data. Using the training data, the outcomes are all patients' survival and the predictors are the first three principal components scores. The fitted coefficient estimates for the three predictors, say \hat{β}₁, \hat{β}₂ and \hat{β}₃, are obtained and used to further scale \tilde{S}ₜ. Finally, the risk scores in raw scale is given by R=\tilde{S}ₜ. (\hat{β}₁, \hat{β}₂, \hat{β}₃)′, and the anti-log 2 transformed risk scores are R₂=2^{R}, where (\hat{β}₁, \hat{β}₂, \hat{β}₃)=(−0.2832, −0.0553, 0.1952). The transformed three one-dimensional risk scores for the gene signature are used to predict the patients that would benefit from ACT. The benefit scores may be generated using the same process. Once the risk scores and benefit scores are generated, the predictive performance of the seven gene signature is then determined by comparing the patients included in a high-risk group or responsive group predicted to benefit from a chemotherapy treatment (e.g., ACT) to the group of patients known (in the validation dataset) to have received a beneficial chemotherapy treatment outcome.

### Example 3

### Normalizing Gene Expression Data

The above calculation assumes that the expression data are normalized. To normalize the expression data, the NanoStringNorm method was employed, which is designed to normalize mRNA and miRNA expression data from the NanoString platform. The normalization is conducted in 3 steps. Assume the p×n matrix denotes the raw read data from n samples measured on p genes. First, the positive controls are used to normalize for technical assay variation. First, the geometric mean p̅ᵢ, for i=1, . . . , n, of the positive control probes is calculated for each sample. Then the mean of these p̅ᵢs, say p̅ are obtained. The method adjusts each sample based on its relative value to all samples. Specifically, the raw read counts of each sample are multiplied by

$$\frac{\overline{\overline{p}}}{{\overline{p}}_{i}} .$$

The second normalization step accounts for the background noise level. Background is calculated as the mean of negative control probes. The calculated background is then subtracted from each sample. The last step normalizes for sample or RNA content by geometric means of housekeeping genes. Similar to the positive controls normalization, the geometric means of the housekeeping genes, h̅ᵢs are calculated for all samples. Denote the mean of h̅ᵢs as h̅. Then the expressions of each sample are multiplied by

$$\frac{\overline{\overline{h}}}{{\overline{h}}_{i}} .$$

Finally, the normalized expressions are log 2 transformed.

Note that p̅ and h̅ also serve as the expected summary values for positive controls and housekeeping genes for new samples. For calculating risk scores from 7- or 12-gene signatures, fixing p̅ and h̅ to the values calculated from the MDACC FFPE data allows for new samples to be normalized to the same levels as the MDACC FFPE data without changing the normalization factors of existing samples. The housekeeping genes in the MDACC FFPE data are ACTB, LRCH1, LYL1, RPLP0, RPS10, RPS16 and RPS19.

### Example 4

### Predictive Performance in Fresh Frozen Samples

The predictive performance of the 7-gene signature was evaluated using two sets of fresh frozen samples collected from patients with non-small cell lung carcinoma (NSCLC). Two datasets with mRNA expression from fresh frozen samples were used to test the predictive performances of the 7-gene signature. The first dataset obtained from the University of Texas Lung Cancer Specialized Program of Research Excellence (SPORE) included 123 patients with adenocarcinomas (ADCs) and 53 patients with squamous cell carcinomas (SCCs); 49 patients received ACT (adjuvant chemotherapy) and 127 patients did not receive ACT. The second dataset (JBR.10 trial, or GSE14814) included 90 samples (49 from patients treated with Vinorelbine plus Cisplatin ACT and 41 patients without ACT).

The analysis of the first dataset shown that the ACT-treated patients showed longer survival than those without ACT (HR 0.376 [0.147-0.965], p=0.0342; FIG. 4A) in the high-risk group; while patients with ACT treatment had no significant survival benefits (HR, 0.725 [0.24-2.19], p=0.569; FIG. 4B) in the low-risk group.

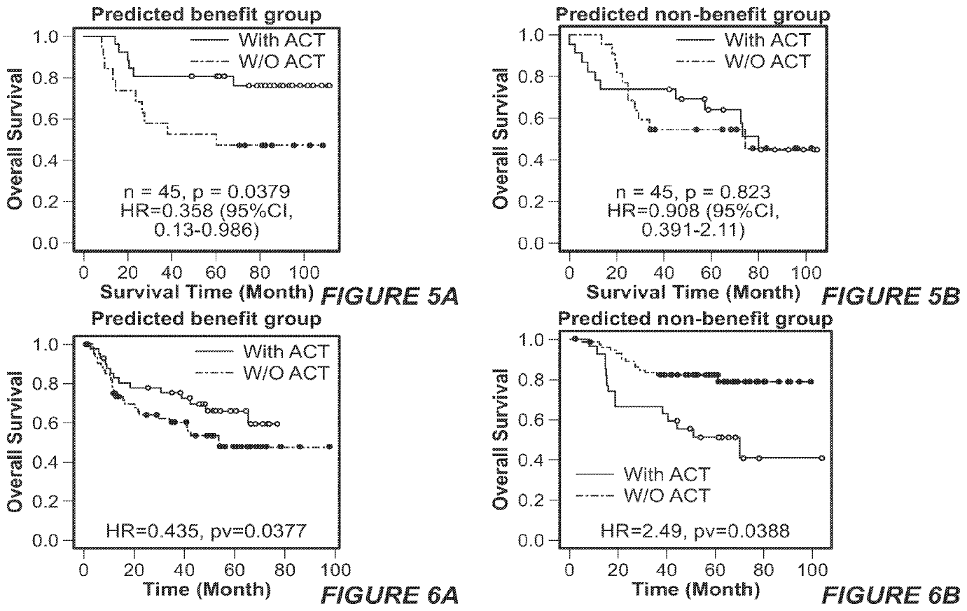

For the GSE14814 dataset, the ACT-treated patients showed longer survival than those without ACT (HR 0.358 [0.13-0.986], p=0.0379; FIG. 5A) in the high-risk group (or predicted benefit group); while patients with ACT treatment had no significant survival benefits (HR, 0.908 [0.391-2.11], p=0.823; FIG. 5B) in the low-risk group (or predicted non-benefit group). Furthermore, the patients with ACT treatment even had worse survival outcomes in the first 21 months for the low-risk group.

### Example 5

### Predictive Performance in FFPE Samples

The predictive performance of the 7-gene signature was evaluated using one set of formalin-fixed paraffin-embedded (FFPE) samples collected from patients with non-small cell lung carcinoma (NSCLC). The FFPE tissue samples of 327 early stage (stage I and II) NSCLC patients were obtained from the University of Texas Lung Cancer Specialized Program of Research Excellence (SPORE) Tissue Bank. Among these 327 early stage patients, 69 (21.1%) were treated with ACT, and the remaining 258 (78.9%) were not treated with ACT. None of the patients received neo-adjuvant chemotherapy. The 258 patients without ACT (166 ADC, 86 SCC and 6 others) were used as the validation cohort for prognostic performance of the 7-gene signature. Since this is a retrospective cohort, in order to minimize the confounding factors, a propensity score matching technique to estimate the effect of ACT by accounting for the covariates was used. From the original 327 patients, a cohort of 207 propensity score-matched patients was derived, among which 69 patients (33.3%) were treated with ACT and 138 patients (66.7%) were not treated with ACT, to validate the predictive performance of the assay in FFPE samples.

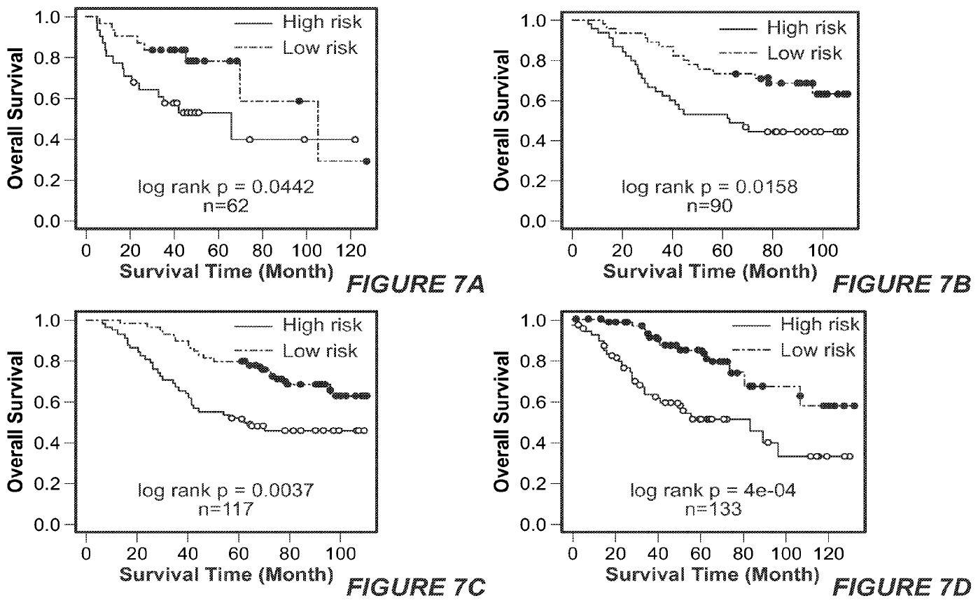

The 207 early stage NSCLC patients (propensity score-matched for ACT treatment) in the validation cohort were placed by the assay into two groups: those predicted to benefit from ACT (ACT benefit) and those predicted not to benefit from ACT (non-benefit) group, using the same risk score and cutoff criteria. FIG. 6 shows the recurrence-free survival curves for patients with and without ACT in the predicted ACT benefit and non-benefit groups, respectively. In the predicted ACT benefit group (high-risk group) (FIG. 6A), the patients who received ACT had longer RFS time than those who did not receive ACT, while in the predicted ACT non-benefit group (low-risk group) (FIG. 6B), patients who received ACT actually exhibited worse survival than those who did not receive ACT.

Multivariate analyses for the efficacy of ACT treatment shown that ACT had significant relapse-free survival (RFS) benefit (HR=0.435, p=0.0377) in the predicted ACT benefit group while ACT was not associated with RFS benefit (HR=2.49, p=0.0388) in the predicted ACT non-benefit group after adjusting for other clinical variables. To test the interaction between the predicted ACT benefit groups and ACT treatment effects, multivariate analysis adjusting for clinical variables were performed, including histology, smoking status, age, gender, tumor size, and stage, for all 207 patients. This analysis indicated that, after adjusting for other clinical variables, there was significant interaction (p=0.000874) between the effect of ACT and the predicted risk groups. The ROC curve for the predictive analysis was calculated.

### Example 6

### Prognosis Performance in Fresh Frozen Samples

The performance of the 7-gene signature was evaluated in four independent datasets measured from fresh frozen tumor samples to assess the prognosis performance of the signature in fresh frozen tumor samples.

In a validation cohort with 62 lung ADC patients, the patients in the predicted high-risk group have significantly worse survival than those in the predicted low-risk group (p=0.0442) (FIG. 7A); in a validation cohort with 90 lung ADC patients, the patients in the predicted high-risk group have significantly worse survival than those in the predicted low-risk group (p=0.0158) (FIG. 7B); in a validation cohort with 117 lung ADC patients, the patients in the predicted high-risk group have significantly worse survival than those in the predicted low-risk group (p=0.0037) (FIG. 7C); in a validation cohort with 133 lung ADC patients, the patients in the predicted high-risk group have significantly worse survival than those in the predicted low-risk group (p=4E-4) (FIG. 7D).

### Example 7

### Prognosis Performance in FFPE Samples

The performance of the 7-gene signature was evaluated in independent dataset measured from FFPE tumor samples to assess the prognosis performance of the signature in FFPE tumor samples.

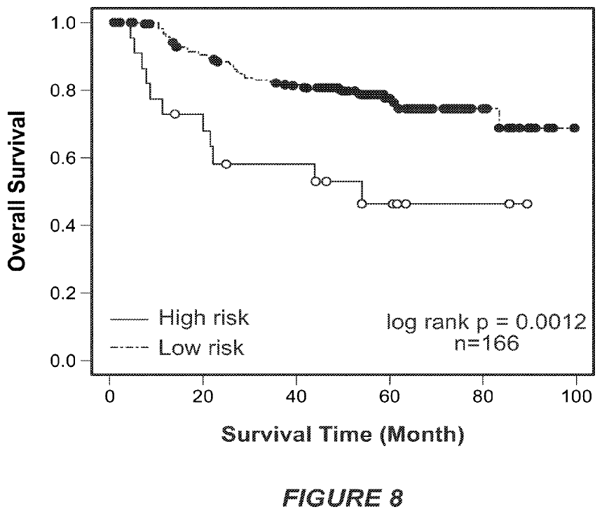

In a validation cohort with 166 early stage lung ADC patients, the patients in the predicted high-risk group have significantly worse survival than those in the predicted low-risk group (p=0.0012) (FIG. 8).

### System Hardware

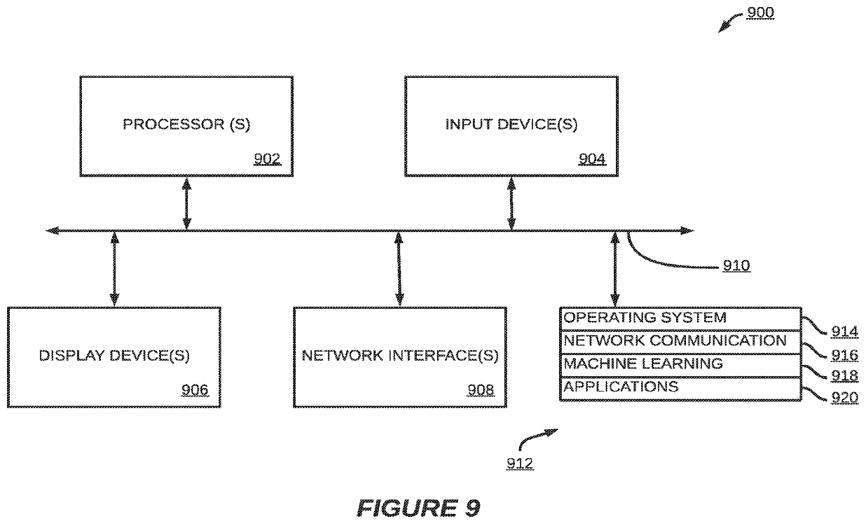

FIG. 9 shows an example computer system according to an embodiment of the present disclosure. The computer system may include a computing device 900 that may implement a machine learning system that predicts cancer progression and/or treatment response in a patient based on a gene signature. The computing device 900 may be implemented on any electronic device that runs software applications derived from compiled instructions, including without limitation personal computers, servers, smart phones, media players, electronic tablets, game consoles, email devices, etc. In some implementations, the computing device 900 may include one or more processors 902, one or more input devices 904, one or more display devices 906, one or more network interfaces 908, and one or more computer-readable mediums 912. Each of these components may be coupled by bus 910, and in some embodiments, these components may be distributed among multiple physical locations and coupled by a network.

Display device 906 may be any known display technology, including but not limited to display devices using Liquid Crystal Display (LCD) or Light Emitting Diode (LED) technology. Processor(s) 902 may use any known processor technology, including but not limited to graphics processors and multi-core processors. Input device 904 may be any known input device technology, including but not limited to a keyboard (including a virtual keyboard), mouse, track ball, camera, and touch-sensitive pad or display. Bus 910 may be any known internal or external bus technology, including but not limited to ISA, EISA, PCI, PCI Express, USB, Serial ATA or FireWire. Computer-readable medium 912 may be any non-transitory medium that participates in providing instructions to processor(s) 902 for execution, including without limitation, non-volatile storage media (e.g., optical disks, magnetic disks, flash drives, etc.), or volatile media (e.g., SDRAM, ROM, etc.).

Computer-readable medium 912 may include various instructions 914 for implementing an operating system (e.g., Mac OS®, Windows®, Linux). The operating system may be multi-user, multiprocessing, multitasking, multithreading, real-time, and the like. The operating system may perform basic tasks, including but not limited to: recognizing input from input device 904; sending output to display device 906; keeping track of files and directories on computer-readable medium 912; controlling peripheral devices (e.g., disk drives, printers, etc.) which can be controlled directly or through an I/O controller; and managing traffic on bus 910. Network communications instructions 3116 may establish and maintain network connections (e.g., software for implementing communication protocols, such as TCP/IP, HTTP, Ethernet, telephony, etc.).

Machine learning instructions 918 may include instructions that enable computing device 900 to function as a machine learning system and/or to train machine learning models, train prediction models, determine benefit scores, determine patient treatment response based on the benefit scores, determine risk scores, determine disease prognosis and/or tumor progression based on the risk scores, and the like as described herein. Application(s) 920 may be an application that uses or implements the processes described herein and/or other processes. The processes may also be implemented in operating system 914. For example, application 920 and/or operating system may create tasks in applications as described herein.

The described features may be implemented in one or more computer programs that may be executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program may be written in any form of programming language (e.g., Objective-C, Java), including compiled or interpreted languages, and it may be deployed in any form, including as a stand-alone program or as a module, component, subroutine, or other unit suitable for use in a computing environment.

Suitable processors for the execution of a program of instructions may include, by way of example, both general and special purpose microprocessors, and the sole processor or one of multiple processors or cores, of any kind of computer. Generally, a processor may receive instructions and data from a read-only memory or a random access memory or both. The essential elements of a computer may include a processor for executing instructions and one or more memories for storing instructions and data. Generally, a computer may also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data may include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory may be supplemented by, or incorporated in, ASICs (application-specific integrated circuits).

To provide for interaction with a user, the features may be implemented on a computer having a display device such as an LED or LCD monitor for displaying information to the user and a keyboard and a pointing device such as a mouse or a trackball by which the user can provide input to the computer.

The features may be implemented in a computer system that includes a back-end component, such as a data server, or that includes a middleware component, such as an application server or an Internet server, or that includes a front-end component, such as a client computer having a graphical user interface or an Internet browser, or any combination thereof. The components of the system may be connected by any form or medium of digital data communication such as a communication network. Examples of communication networks include, e.g., a telephone network, a LAN, a WAN, and the computers and networks forming the Internet.

The computer system may include clients and servers. A client and server may generally be remote from each other and may typically interact through a network. The relationship of client and server may arise by virtue of computer programs running on the respective computers and having a client-server relationship to each other.

One or more features or steps of the disclosed embodiments may be implemented using an API. An API may define one or more parameters that are passed between a calling application and other software code (e.g., an operating system, library routine, function) that provides a service, that provides data, or that performs an operation ora computation.

The API may be implemented as one or more calls in program code that send or receive one or more parameters through a parameter list or other structure based on a call convention defined in an API specification document. A parameter may be a constant, a key, a data structure, an object, an object class, a variable, a data type, a pointer, an array, a list, or another call. API calls and parameters may be implemented in any programming language. The programming language may define the vocabulary and calling convention that a programmer will employ to access functions supporting the API.

In some implementations, an API call may report to an application the capabilities of a device running the application, such as input capability, output capability, processing capability, power capability, communications capability, etc.

While various embodiments have been described above, it should be understood that they have been presented by way of example and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments. For example, other steps may be provided, or steps may be eliminated, from the described flows, and other components may be added to, or removed from, the described systems. Accordingly, other implementations are within the scope of the following claims.

In addition, it should be understood that any figures which highlight the functionality and advantages are presented for example purposes only. The disclosed methodology and system are each sufficiently flexible and configurable such that they may be utilized in ways other than that shown.

Although the term “at least one” may often be used in the specification, claims and drawings, the terms “a”, “an”, “the”, “said”, etc. also signify “at least one” or “the at least one” in the specification, claims and drawings.

Finally, it is the applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

Although the present invention has been described with reference to specific details of certain embodiments thereof in the above examples, it will be understood that modifications and variations are encompassed within the spirit and scope of the invention. Accordingly, the methods and compositions are limited only by the following claims.

### REFERENCES

1. Tang H, Xiao G, Behrens C, et al. A 12-gene set predicts survival benefits from adjuvant chemotherapy in non-small cell lung cancer patients. Clin Cancer Res. 2013; 19(6):1577-1586.
2. Bair, E. H. (2006). Prediction by supervised principal components. Journal of the American Statistical Association, 101(473), 119-137.
3. Tang, H. X. (2013). A 12-Gene Set Predicts Survival Benefits from Adjuvant Chemotherapy in Non-Small Cell Lung Cancer Patients. Clin Cancer Res, 19(6), 1577-1586.

What is claimed is:

1. A method of predicting disease prognosis in of a human subject diagnosed with lung cancer comprising;
resecting a carcinoma tumor of the lung to obtain a lung cancer sample;
obtaining expression information for the seven gene signature in the lung cancer sample obtained from said subject:
normalizing the expression information to generate normalized expression information;
determining a risk score based on the normalized expression information;
comparing the risk score to a pre-determined risk threshold; and
determining, the human subject has a high risk of tumor progression based on the comparing the risk score to the pre-determined risk threshold.

2. The method of claim 1, further comprising treating the human subject with the chemotherapy treatment in response to determining the human subject has a high risk of tumor progression.

3. The method of claim 1, wherein the seven gene signature includes RRM2, AURKA, NKX2-1, COL4A3, ATP8A1, C1orf116, and HSD17B6.

4. The method of claim 1, further comprising:
identifying a set of 12 genes that predicts a likelihood that the human subject will benefit from a chemotherapy treatment;
calculating a regression coefficient for each gene included in the set of 12 genes;
determining a regression value threshold using a cross validation process; and
identifying the seven gene signature from the set of 12 genes based on a comparison of the regression coefficient for each gene to the regression value threshold.

5. The method of claim 1, wherein the normalized expression information adjusts the expression information for technical assay variation, background noise, and RNA content.

6. The method of claim 1, further comprising
generating a validation dataset including expression information obtained from a plurality of fresh frozen lung cancer samples;
determining three principal component scores for each gene included in the seven gene signature based on the expression information;
combining the three principal component scores for each gene to generate a one-dimensional risk score for each principal component; and
comparing a high-risk group of patients predicted to benefit from a chemotherapy treatment based on the one-dimensional risk scores to a group of patients having a beneficial chemotherapy treatment outcome included in the validation dataset.

7. A method of predicting a response to an adjuvant chemotherapy (ACT) in a subject diagnosed with lung cancer comprising:
determining expression levels of the following genes: RRM2, AURKA, NKX2-1, COL4A3, ATP8A1, C1orf116, and HSD1766, in a lung cancer sample obtained from the subject, wherein an alteration in the expression of the genes, as compared to an average expression in lung cancer, indicates that the subject will respond favorably to adjuvant chemotherapy.

8. The method of claim 7, further comprising treating the subject with one or more adjuvant chemotherapies.

9. The method of claim 1 or 7, wherein the lung cancer sample is a fresh frozen tumor (FF) sample.

10. The method of claim 1 or 7, wherein the lung cancer sample is a formalin-fixed paraffin-embedded (FFPE) tumor sample.

11. The method of claim 1 or 7, wherein the lung cancer is non-small cell lung cancer (NSCLC), lung adenocarcinoma (ADC), or lung squamous cell carcinoma (SCC).

12. The method of claim 7, wherein the one or more adjuvant chemotherapies are vinorelbine, cisplatin, carboplatin, gemcitabine, paclitaxel, topotecan, docetaxel, irinotecan, pemetrexed, etoposide, or any combination thereof.

13. The method of claim 7, wherein administering an ACT to the subject improves survival of the subject.

14. The method of claim 7, further comprising:
determining a risk score of the subject by combining expression information of the genes, and
predicting a risk group of the subject by comparing the risk score of the subject to a one-dimensional risk score.

15. A method of predicting a response to chemotherapy of a human subject diagnosed with lung cancer comprising;
resecting a carcinoma tumor of the lung to obtain a lung cancer sample;
obtaining expression information for the seven gene signature in the lung cancer sample obtained from said subject:
normalizing the expression information to generate normalized expression information;
determining a benefit score based on the normalized expression information;
comparing the benefit score to a pre-determined benefit threshold; and
determining, a level of chemotherapy responsiveness for human subject based on the comparing the benefit score to the pre-determined benefit threshold.

16. The method of claim 15, further comprising determining the human subject will benefit from chemotherapy treatment based on the level of chemotherapy responsiveness; and
administering to the human subject a chemotherapy treatment.

17. The method of claim 15, further comprising determining the human subject will not benefit from chemotherapy treatment based on the level of chemotherapy responsiveness; and
administering to the human subject at least one of an immunotherapy or targeted therapy.

18. The method of claim 16, further comprising;
performing an observational follow up on the patient, the observational follow up including an analysis of expression information for the seven gene signature in a second lung cancer sample obtained from the patient.

19. The method of claim 15, wherein the seven gene signature includes RRM2, AURKA, NKX2-1, COL4A3, ATP8A1, C1orf116, and HSD17B6.

20. The method of claim 1, wherein the normalized expression information adjusts the expression information for technical assay variation, background noise, and RNA content.
